(19) United States
(12) Patent Application Publication
(10) Pub. No.: US 2005/0149110 A1
(43) Pub. Date: Jul. 7, 2005

(54) Vascular catheter with an expandable section and a distal tip for delivering a thromboembolic protection device and method of use

(76) Inventors: Mark H. Wholey, Oakmont, PA (US); Michael H. Wholey, San Antonio, TX (US)

Correspondence Address: Lara A. Northrop;Pietragallo, Bosick & Gordon, One Oxford Centre, 38th Floor, 301 Grant Street, Pittsburgh, PA, 15219, US

(21) Appl. No.: 11/014,496

(22) Filed: Dec. 16, 2004

Related U.S. Application Data
(60) Provisional application No. 60/529,846, filed on Dec. 16, 2003.

(51) Int. Cl. A61M 29/00

(52) U.S. Cl. 606/200

(57) ABSTRACT

A vascular catheter including a radially expandable initial dilation segment, such as an inflatable balloon and an expanded distal tip for releasably housing at least a portion of a thromboembolic protection device is disclosed. A vascular catheter including a shaft structured and arranged to at least partially house a thromboembolic protection device, a radially expandable initial dilation segment disposed on the shaft, and a radially expandable stent expanding segment disposed on the shaft, is also disclosed.

### RELATED APPLICATIONS

The present application claims the benefit of U.S. Provisional Patent Application Ser. No. 60/529,846 filed Dec. 16, 2003, which is incorporated herein by reference.

### FIELD OF THE INVENTION

The present invention relates to vascular catheters, and more particularly relates to a vascular catheter having a shaft including a distal tip having an interior structured an arranged to releasably house a thromboembolic protection device, and an initial dilation segment disposed on the shaft.

### BACKGROUND INFORMATION

It is common practice today to open occluded (i.e. blocked) or stenotic (i.e. narrowed) blood vessels by inserting a guide wire, then inserting a catheter carrying a balloon shaped segment and subsequently inflating the balloon, which exerts a radial force to press stenosis outward against the wall of the blood vessel. This procedure is called balloon angioplasty. Frequently, an implantable metallic stent will also be used to provide greater radial strength at the stenotic portion of the blood vessel, and to provide longer-term patency.

In order to help deliver balloon catheters and stent devices, special guiding catheters or sheaths are often used. These guiding catheters or sheaths are placed away (or upstream) from the targeted lesion or stenotic area. A guide wire may be advanced past the stenotic area, allowing balloon catheters and stents to be advanced through the guiding catheter or sheath to the target area of the blood vessel.

During the balloon angioplasty procedure and stent placement at the stenotic lesion, there is a risk of dislodging fragments of plaque, thrombus (blood clots) and/or other material. These fragments may become dislodged from the stenotic lesion when the balloon segment is inflated. If the lesion involves arterial circulation, then the dislodged particles could flow into smaller vessels in the brain, other organs, and/or extremities, resulting in significant complications. Likewise, if the lesions involve the venous circulation, then the dislodged fragments could flow into the heart and lungs again resulting in serious complications. Embolic protection devices are typically used to provide protection from such dislodged fragments of plaque and thrombus. These protection devices often consist of a small umbrella-like filter or lasso-shaped device attached to the end of a guide wire.

Traditional methods used to open occluded or stenotic blood vessels typically require at least a five-step process. The first step can involve the insertion of a delivery sheath housing a thromboembolic protection device in a collapsed position along a guide wire to an area upstream of a restricted blood vessel, expanding the thromboembolic protection device to an open position and removing the delivery sheath along the guide wire. The second step typically involves the delivery of a plaque-smoothing balloon along the guide wire to the occluded or stenotic area of the blood vessel, inflating the balloon to an expanded position, subsequently deflating the balloon and removing the balloon from the occluded area along the guide wire. The third step typically involves delivering a stent to the occluded area of the blood vessel along the guide wire. The fourth step typically involves the delivery of a stent-expanding balloon along the guide wire to an area inside the stent within the occluded area of the blood vessel, expanding the balloon within the interior of the stent to an expanded position, deflating the balloon and removing the balloon along the guide wire. The fifth step typically involves the insertion of a retrieval sheath along the guide wire to an area in close proximity to the thromboembolic protection device, collapsing the thromboembolic protection device to a contracted position, inserting the thromboembolic protection device into the recovery sheath and removing the recovery sheath, thromboembolic protection device and guide wire from the blood vessel.

A significant disadvantage to the present system of filter delivery and predilation is that it includes multiple steps. In carotid stent placement, longer procedure time and the more steps that are involved, are directly related to increased chance of complication. A need exists for a catheter that serves the dual purpose of providing balloon angioplasty to a stenotic lesion of a blood vessel, while at the same time providing an effective means for safely advancing, deploying and providing the means to expand an embolic protection filter or other device which will capture and contain dislodged plaque and thromboembolic material.

The present invention has been developed in view of the foregoing, and to address other deficiencies of the prior art.

### SUMMARY OF THE INVENTION

An aspect of the present invention is to provide a vascular catheter for delivery of a thromboembolic protection device within a blood vessel of a patient comprising a shaft including an expanded distal tip having an interior structured and arranged to releasably house at least a portion of the thromboembolic protection device within the interior of the distal tip during delivery of the thromboembolic protection device, and a radially expandable initial dilation segment disposed on the shaft.

Another aspect of the present invention is to provide a vascular catheter for delivery of a thromboembolic protection device in a blood vessel of a patient comprising means for releasably housing at least a portion of the thromboembolic protection device during delivery of the thromboembolic protection device, and means for initially dilating at least a portion of a stenoted and/or occluded area of the blood vessel.

Another aspect of the present invention is to provide a method of dilating a blood vessel, comprising inserting into the blood vessel a vascular catheter comprising a shaft, a thromboembolic protection device housed at least partially within a distal tip of the shaft, and a radially expandable initial dilation segment, expanding the thromboembolic protection device from the shaft from a collapsed position to an expanded position, and expanding the radially expandable initial dilation segment to dilate at least a portion of a stenoted and/or occluded area of the blood vessel.

Another aspect of the present invention is to provide a vascular catheter comprising a shaft structured and arranged to at least partially house a thromboembolic protection device, a radially expandable initial dilation segment disposed on the shaft, and a radially expandable stent expanding segment disposed on the shaft.

Another aspect of the present invention is to provide a vascular catheter comprising a shaft including means for initially dilating a portion of a stenoted and/or occluded area of the blood vessel, and means for radially expanding a stent disposed about the shaft.

Yet another aspect of the present invention is to provide a method of dilating a blood vessel comprising inserting into the blood vessel a vascular catheter comprising a shaft housing a thromboembolic protection device, a radially expandable initial dilation segment disposed on the shaft, and a radially expandable stent expanding segment disposed on the shaft, expanding the thromboembolic protection device from the shaft, expanding the radially expandable initial dilation segment to dilate at least a portion of a stenoted and/or occluded area of the blood vessel, and expanding the radially expandable stent expanding segment to expand a stent against the stenoted and/or occluded area of the blood vessel.

These and other aspects of the present invention will be more apparent from the following description.

### BRIEF DESCRIPTION OF THE DRAWINGS

FIG. 1 is a partially schematic longitudinal sectional view of a catheter apparatus in accordance with an embodiment of the present invention.

FIG. 2 is a longitudinal side view of the apparatus of FIG. 1.

FIG. 3 is a sectional view taken along the line 3-3 of the apparatus of FIG. 2.

FIG. 4 is a longitudinal side view of a catheter apparatus in accordance with an embodiment of the present invention.

FIG. 5 is a sectional view taken along the line 4-4 of the apparatus of FIG. 4.

FIG. 6 is a sectional view taken along the line 3-3 of the apparatus of FIG. 4.

FIG. 7 is a partially schematic longitudinal side view of a catheter apparatus in accordance with another embodiment of the present invention.

FIG. 8 is a sectional view taken along the line 11-11 of the apparatus of FIG. 7.

FIG. 9 is a sectional view taken along the line 12-12 of the apparatus of FIG. 7.

FIG. 10 is a partially schematic longitudinal side view of a catheter apparatus in accordance with another embodiment of the present invention.

FIG. 11 is a sectional view taken along the line 14-14 of the apparatus of FIG. 10.

FIG. 12 is a sectional view taken along the line 15-15 of the apparatus of FIG. 10.

FIG. 13 is a sectional view taken along the line 16-16 of the apparatus of FIG. 10.

FIG. 14 is a longitudinal side view or the apparatus or FIG. 1 shown in conjunction with a guide wire and a thromboembolic protection device mounted on the guide wire in accordance with an embodiment of the present invention.

FIG. 15 is a longitudinal side view of the apparatus of FIG. 1 shown in conjunction with a guide wire and a thromboembolic protection device mounted on the guide wire, with the thromboembolic protection device being partially advanced from the distal end or advanced from the distal end of the catheter apparatus in accordance with an embodiment of the present invention.

FIG. 16 is a longitudinal side view of the apparatus of FIG. 1 shown in conjunction with a guide wire and a thromboembolic protection device mounted on the guide wire, with the thromboembolic protection device delivered from the distal end of the catheter apparatus in accordance with an embodiment of the present invention.

FIG. 17 is a longitudinal side view of the apparatus of FIG. 1, with the balloon segment in a deflated position in accordance with an embodiment of the present invention.

FIG. 18 is a partially schematic longitudinal sectional view of a proximal end of a catheter apparatus in accordance with an embodiment of the present invention.

FIG. 19 shows a tandem balloon arrangement of the present invention.

FIG. 20 shows a dual-stage balloon arrangement of the present invention.

FIG. 21 shows the apparatus of FIG. 1 being used to treat a stenosis of a blood vessel. FIG. 21 also shows the apparatus of FIG. 1 being used in conjunction with a guide wire and a thromboembolic protection device mounted on the guide wire, and an initial dilation segment being used to smooth a stenotic region of a blood vessel in accordance with an embodiment of the present invention.

FIG. 22 shows the apparatus of FIG. 1 being used to treat a stenosis of a blood vessel in accordance with an embodiment of the present invention. FIG. 22 shows that the inflated initial dilation segment has smoothed the stenotic region of the blood vessel in accordance with an embodiment of the present invention.

FIG. 23 shows that a stent has been placed in the stenotic portion of the blood vessel in accordance with an embodiment of the present invention.

FIG. 24 shows the apparatus of FIG. 1 being used to treat a stenosis of a blood vessel in accordance with an embodiment of the present invention. FIG. 24 shows that the stent-expanding segment has been expanded to cause the stent to assume its expanded position.

### DETAILED DESCRIPTION OF THE INVENTION

The present invention provides a sheath or catheter capable of housing a thromboembolic protection device in a distal tip and at least one radially expandable segment, such as an inflatable balloon, disposed on the shaft of the sheath. In one embodiment, the apparatus of the present invention can be used in conjunction with the catheter apparatus described in U.S. Patent Application Publication No. 2003/0176886, which is incorporated herein by reference.

As used herein, the term “thromboembolic protection device” includes filters, strainers, lassos, nets, traps, and/or any other assembly or device capable of capturing embolic material during an interventional procedure such as transluminal angioplasty or stenting. As used herein, the term “embolic material” includes plaque, thrombus, thromboembolic fragments, and/or any other material that may be dislodged from a blood vessel or released into the blood stream during an interventional procedure such as transluminal angioplasty.

At least a portion of the thromboembolic protection device is releasably housed within the sheath during delivery of the thromboembolic protection device to an area downstream of an occluded and/or stenotic region of a blood vessel of a patient. As used herein, the term “releasably housed” means that at least a portion of the thromboembolic protection device can be stored, in a collapsed position, within the interior of the sheath during delivery of the thromboembolic protection device, and subsequently deployed to achieve an expanded position upon completion of delivery. As used herein, the term “completion of delivery” means that at least a portion of the thromboembolic protection device is positioned at least partially downstream of an occluded or stenotic area of a blood vessel.

As used herein, the term “downstream of an occluded or stenotic area” means an area within a blood vessel that blood will flow to after passing through an area that is occluded, stenotic and/or otherwise restricted. As used herein, the term “patient” includes both humans and animals.

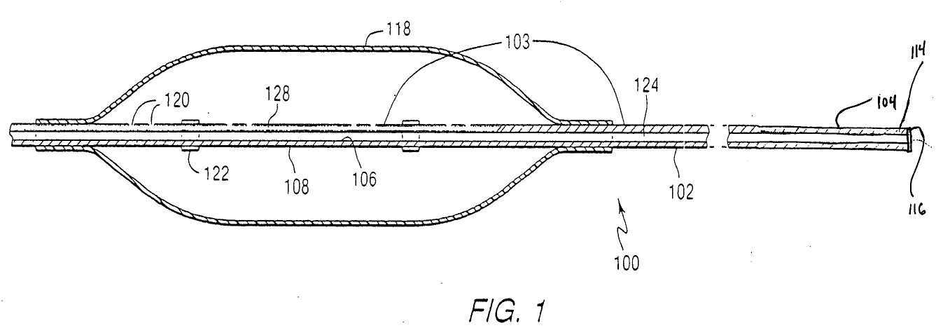

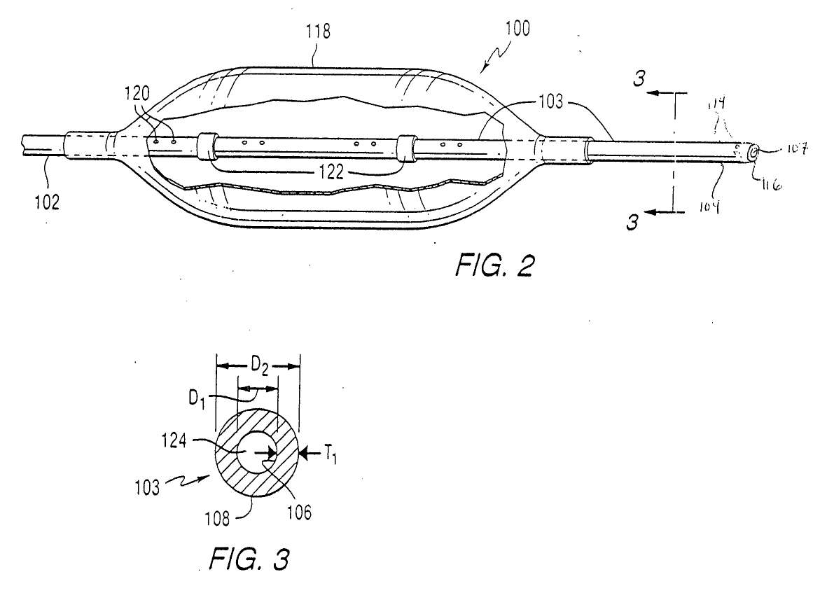

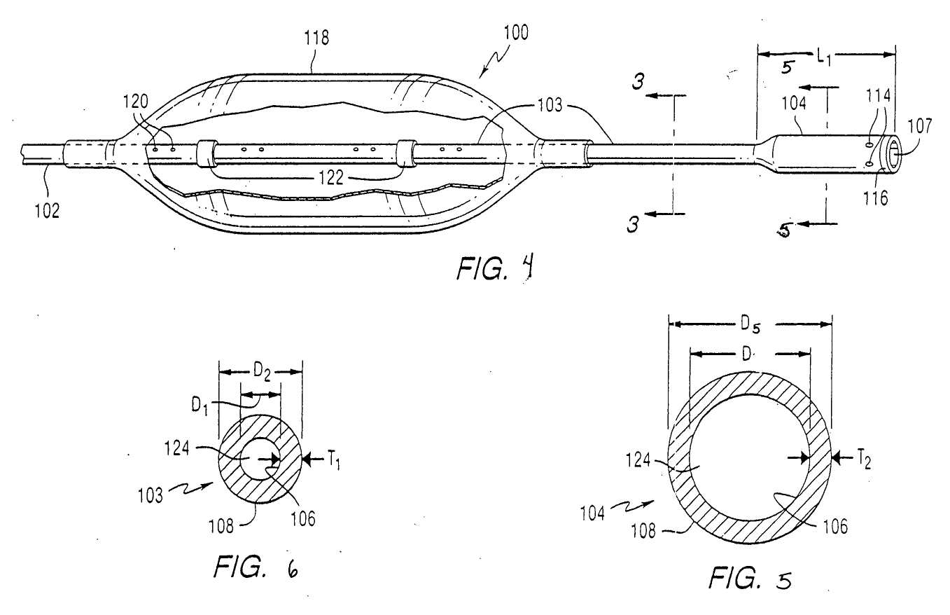

FIGS. 1 and 2 show a partially schematic longitudinal sectional view of a delivery sheath or catheter 100 in accordance with an embodiment of the present invention. The sheath 100 may include a shaft 102, and the shaft includes an intermediate portion 103 and a distal tip 104. The shaft of the catheter apparatus 100 may be made out of any suitable material, such as polyethylene, polyamide, polytetraflurethylene, or any other polyester compounds. The sheath or catheter may include a distal tip having the same diameter as the body of the catheter. In this embodiment, the distal tip may be substantially cylindrical shaped, as shown in FIGS. 1 and 2. FIGS. 1 and 2 show that the shaft 102 includes inner wall 106 and outer wall 108. FIGS. 2 and 4 also show that the distal tip 104 may include one or more tip apertures 114 running radially outward from the inner wall 106 of the shaft 102 to the outer wall 108 of the shaft, which may be used to receive various diagnostic instruments and/or for aspirating embolic debris. The distal tip 104 may optionally include a soft and substantially flexible atraumatic material 116 near the distal end 107 of the distal tip 104. The atraumatic material 116 may be made out of any suitable material, such as polyethyltetrafluride (PET), polytetraflurethylene (PTFE), polyamide, or any other polyester compounds. This atraumatic portion 116 of the distal tip 104 may optionally be coated or constructed with a material of higher atomic density to aid in visualizing the distal tip 104, for instance, under fluoroscopy.

FIG. 3 is a cross-sectional view of the intermediate portion 103 of the shaft 102 of the sheath apparatus 100 shown in FIG. 2 taken along the line 3-3. The cross section of the sheath or catheter 100 shown in FIG. 3 may have an inner diameter D₁ defined and measured with respect to the inner wall 106 of the shaft 102, an outer diameter D₂ defined and measured with respect to the outer wall 108 of the shaft 102, and a thickness T₁ defined as the distance between the inner wall 106 and the outer wall 108 of the shaft 102. The inner diameter D₁ may range from about 0.4 mm to about 0.6 mm, preferably from about 0.45 mm to about 0.55 mm. A particularly preferred diameter D₁ may be about 0.48 mm. The outer diameter D₂ may range from about 0.9 mm to about 3 mm, preferably from about 1.5 mm to about 3 mm. A particularly preferred diameter D₂ may be about 2.5 mm. The thickness T₁ may range from about 0.25 mm to about 1.2 mm, preferably from about 0.5 mm to about 1.2 mm. A particularly preferred thickness T₁ may be about 1 mm. Although a particular cross-sectional piece of the intermediate portion 103 of the catheter shaft 102 is shown in FIG. 3, it is to be understood that the diameters D₁ and D₂, and the thickness T₁ may be measured at other locations along the intermediate portion of the catheter shaft, such as the portion of the shaft 102 containing the inflatable balloon segment 118. The first lumen 124 and the second lumen 128 are both illustrated in the cross section of the catheter 100 shown in FIG. 3.

In another embodiment, the sheath or catheter may have an expanded distal tip. As used herein, the term “expanded distal tip” means a distal portion of a shaft of a sheath or catheter that has a larger interior storage volume when compared to the interior storage volume of a proximal portion of the shaft. The thromboembolic protection device can be releasably housed within the distal tip of the sheath.

As shown in FIGS. 4, 7 and 10, when the distal tip 104, 204 and 304 is an expanded distal tip, the expanded distal tip 104 has a cross-sectional diameter measured with respect to the inner wall 106 that is greater than a cross-sectional diameter of the intermediate portion of the shaft 103 measured with respect to the inner wall 106. Also shown in FIGS. 4, 7 and 10, the expanded distal tip 104 has a cross-sectional diameter measured with respect to the outer wall 108 that is greater than a cross-sectional diameter of the intermediate portion of the shaft 103 measured with respect to the outer wall 108.

FIG. 5 shows a cross-sectional portion of the expanded distal tip 104 of the catheter 100 shown in FIG. 4 taken along the line 4-4. As shown in FIG. 5, an inner diameter D₃ may be defined and measured with respect to the inner wall 106 of the shaft 102, an outer diameter D₄ may be defined and measured with respect to the outer wall 108 of the shaft 102, and a thickness T₂ may be defined as the distance between the inner wall 106 and the outer wall 108 of the shaft 102. The inner diameter D₃ may range from about 0.8 mm to about 1.2 mm, preferably from about 0.95 mm to about 1.1 mm. A particularly preferred diameter D₃ may be about 1 mm. The outer diameter D₄ may range from about 1.3 mm to about 3.6 mm, preferably from about 2 mm to about 3.3 mm. A particularly preferred diameter D₄ may be about 3 mm. The thickness T₂ may range from about 0.25 mm to about 1.2 mm, preferably from about 0.5 mm to about 1.1 mm. A particularly preferred thickness T₂ may be about 1.0 mm. The first lumen 124 is also shown in FIG. 5.

FIGS. 5 and 6 illustrate that the inner cross-sectional diameter D₃ of the expanded distal tip 104 is greater than the inner cross sectional diameter D₁ of the intermediate portion 103 of the shaft 102, and that the outer cross-sectional diameter D₄ of the expanded distal tip 104 is greater than the outer cross-sectional diameter D₂ of the intermediate portion 103 of the shaft 102. FIGS. 5 and 6 also illustrate that the thickness T₂ is substantially equal to the thickness T₁.

In this embodiment, a ratio of the diameter D₃ to D₁ may be defined as D₃:D₁. D₃:D₁ may range from about 1.6:1 to about 2.4:1, preferably from about 1.8:1 to about 2.2:1. In a particularly preferred embodiment, D₃:D₁ may be about 2:1. In this embodiment, a ratio of the diameter D₄ to the diameter D₂ may also be defined as D₄:D₂. D₄:D₂ may range from about 1.1:1 to about 1.4:1, preferably from about 1.1:1 to about 1.3:1. In a particularly preferred embodiment, D₄:D₂ may be about 1.2:1.

FIG. 4 shows that the length of the expanded distal tip 104 may be defined as L₁. The length L₁ may range from about 0.3 cm to about 1 cm, preferably from about 0.5 cm to about 0.7 cm. A particularly preferred length L₁ may be about 0.7 cm. In this embodiment, a ratio of the diameter D₃ of the expanded distal tip 104 to the length L₁ of the expanded distal tip 104 may be defined as D₃:L₁. D₃:L₁ may range from about 0.27:1 to about 0.12:1, preferably from about 0.19:1 to about 0.13:1. In a particular embodiment, D₃:L₁ may be about 0.14:1. In this embodiment, a ratio of the diameter D₄ of the expanded distal tip 104 to the length L₁ of the distal tip 104 may be defined as D₄:L₁. D₄:L₁ may range from about 0.44:1 to about 0.36:1, preferably from about 0.4:1 to about 0.39:1. In a particular embodiment of the invention, D₄:L₁ may be about 0.4:1.

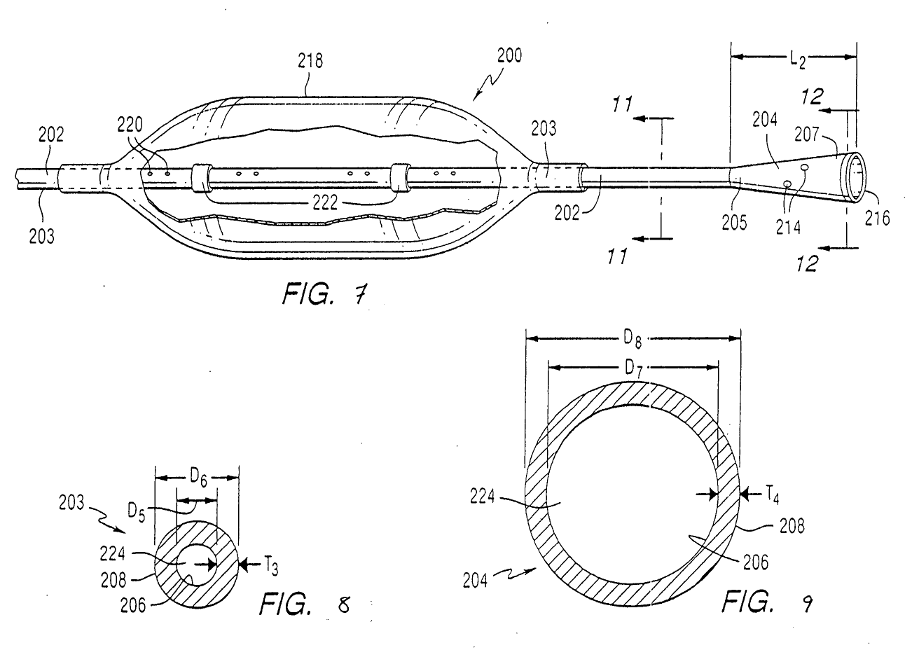

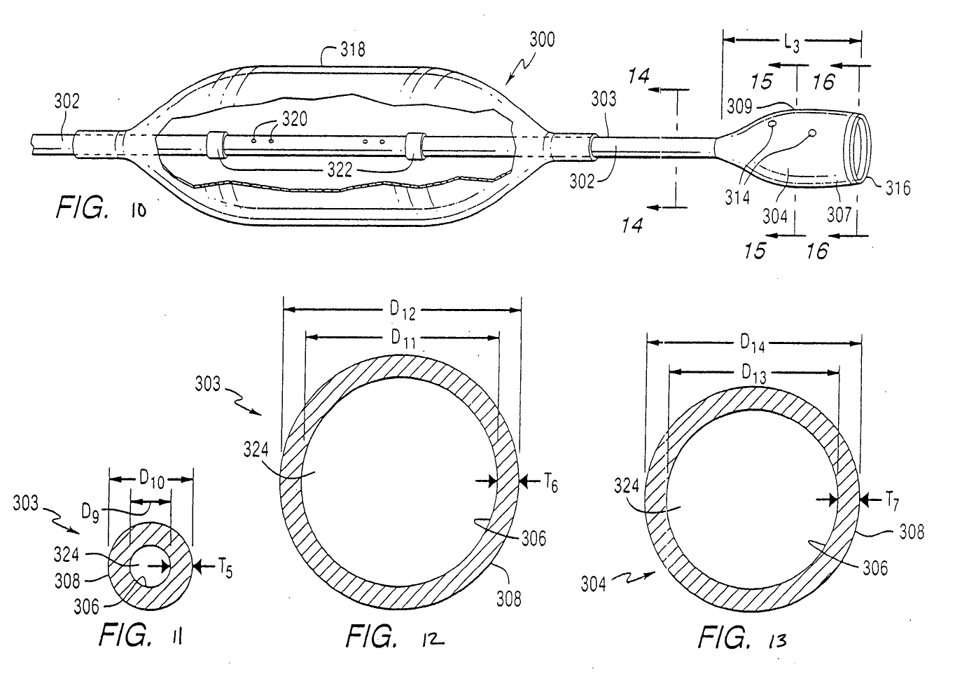

FIGS. 7-9 show a sheath or catheter apparatus 200 having an expanded distal tip in accordance with another embodiment of the present invention. The sheath 200 includes a shaft 202, and the shaft includes an intermediate portion 203 and an expanded distal tip 204. In this embodiment the distal tip may be substantially conical shaped, as shown in FIG. 7, with the diameter of the distal tip 204 gradually increasing towards the distal end 207 of the distal tip 204. FIG. 7 shows that the distal tip 204 may include one or more tip apertures 214 running radially outward from an inner wall 206 of the shaft to an outer wall 208 of the shaft, which may be used for diagnostic purposes such as aspirating or removing thromboembolic material or other particles from a blood vessel. The distal tip 204 may optionally include a soft and substantially flexible atraumatic material 216 near the distal end 207 of the distal tip 204. This atraumatic material 216 may optionally be coated or constructed with a material of higher atomic density to aid in visualizing the distal tip 204, for instance, under fluoroscopy.

FIG. 7 shows that the catheter apparatus 200 may include a radially expandable segment, such as an inflatable balloon segment 218, disposed on the intermediate portion 203 of the shaft 202. FIG. 7 also shows that the intermediate portion 203 of the shaft 202 containing the inflatable balloon segment 218 may include one or more shaft apertures 220 to allow for the inflatable balloon segment 218 to be inflated and/or deflated. The intermediate portion 203 of the shaft 202 containing inflatable balloon segment 218 may also include one or more radiopaque markers 222 constructed with a material of higher atomic density to help show the location of the inflatable balloon segment 218 on the shaft 202.

The shaft 202 also includes an interior cavity defining a first lumen 224, of which a cross-sectional portion is shown in FIGS. 8 and 9, running inside the catheter substantially from a proximal end (not shown) of the catheter and extending substantially to the distal tip 204 of the catheter 200. The first lumen 224 may be used to accommodate guide wires and/or other diagnostic devices or instruments. The shaft 202 also may include an interior cavity defining a second lumen 228 running adjacent to the first lumen 224 substantially from the proximal end (not shown) of the catheter 200 and extending substantially to the intermediate portion 203 of the shaft 202 containing the inflatable balloon segment 218. This second lumen 228 may be used, for example, to provide gases, liquids, or other materials via the shaft apertures 220 to the inflatable balloon segment 218 for the purposes of inflating or deflating the balloon segment.

FIG. 8 is a cross-sectional view of the intermediate portion 203 of the shaft 202 of the catheter apparatus 200 shown in FIG. 7 taken along the line 11-11. FIG. 8 shows that the shaft 202 includes inner wall 206 and outer wall 208. The cross section of the catheter 200 shown in FIG. 8 may have an inner diameter D₅ defined and measured with respect to the inner wall 206 of the shaft 202, an outer diameter D₆ defined and measured with respect to the outer wall 208 of the shaft 202, and a thickness T₃ defined as the distance between the inner wall 206 and the outer wall 208 of the shaft 202. The inner diameter Ds may range from about 0.4 mm to about 0.6 mm, preferably from about 0.45 mm to about 0.55 mm. A particularly preferred inner diameter D₅ may be about 0.48 mm. The outer diameter D₆ may range from about 0.9 mm to about 3 mm, preferably from about 1.5 mm to about 3 mm. A particularly preferred outer diameter D₆ may be about 2.5 mm. The thickness T₃ may range from about 0.25 mm to about 1.2 mm, preferably from about 0.5 mm to about 1.2 mm. A particularly preferred thickness T₃ may be about 1 mm. Although a particular cross-sectional piece of the intermediate portion 203 of the catheter shaft 202 is shown in FIG. 13, it is to be understood that the diameters D₅ and D₆, and the thickness T₃ may be measured at other locations along the intermediate portion of the catheter shaft, such as the intermediate portion 203 of the shaft 202 containing the inflatable balloon segment 218. The first lumen 224 is illustrated in the cross section of the shaft 202 shown in FIG. 8.

FIG. 9 shows a cross-sectional portion of the expanded distal tip 204 of the catheter 200 shown in FIG. 7 taken along the line 12-12. An inner diameter D₇ may be defined and measured with respect to the inner wall 206 of the shaft 202, an outer diameter D₈ may be defined and measured with respect to the outer wall 208 of the shaft 202, and a thickness T₄ may be defined as the distance between the inner wall 206 and the outer wall 208 of the shaft 202. The inner diameter D₇ may range from about 0.8 mm to about 1.2 mm, preferably from about 0.95 mm to about 1.1 mm. A particularly preferred diameter D₇ may be about 1 mm. The outer diameter D₈ may range from about 1.3 mm to about 3.6 mm, preferably from about 2 mm to about 3.3 mm. A particularly preferred diameter D₈ may be about 3 mm. The thickness T₄ may range from about 0.25 mm to about 1.2 mm, preferably from about 0.5 mm to about 1.1 mm. A particularly preferred thickness T₄ may be about 1 mm. The first lumen 224 is also shown in FIG. 9.

FIGS. 8 and 9 illustrate that the inner cross-sectional diameter D₇ of the expanded distal tip 204 is greater than the inner cross-sectional diameter D₅ of the intermediate portion 203 of the shaft 202, and that the outer cross-sectional diameter D₈ of the expanded distal tip 204 is greater than the outer cross-sectional diameter D₆ of the intermediate portion 203 of the shaft 202. FIGS. 8 and 9 also illustrate that the thickness T₄ is substantially equal to the thickness T₃.

In this embodiment, a ratio of the diameter D₇ to D₅ may be defined as D₇:D₅. D₇:D₅ may range from about 1.6:1 to about 2.4:1, preferably from about 1.8:1 to about 2.2:1. In a particularly preferred embodiment, D₇:D₅ may be about 2:1. In this embodiment, a ratio of the diameter D₈ to the diameter D₆ may also be defined as D₈:D₆. D₈:D₆ may range from about 1.1:1 to about 1.4:1, preferably from about 1.1:1 to about 1.3:1. In a particularly preferred embodiment, D₈:D₆ may be about 1.2:1.

FIG. 7 shows that the length of the expanded distal tip 204 may be defined as L₂. The length L₂ may range from about 0.3 cm to about 1 cm, preferably from about 0.5 cm to about 0.7 cm. A particularly preferred length L₂ may be about 0.7 cm. In this embodiment, a ratio of the diameter D₇ of the distal tip 204 to the length L₂ of the distal tip 204 may be defined as D₇:L₂. D₇:L₂ may range from about 0.27:1 to about 0.12:1, preferably from about 0.19:1 to about 0.13:1. In a particular embodiment, D₇:L₂ may be about 0.14:1. In this embodiment, a ratio of the diameter D₈ of the distal tip 204 to the length L₂ of the distal tip 204 may be defined as D₈:L₂. D₈:L₂ may range from about 0.44:1 to about 0.36:1, preferably from about 0.4:1 to about 0.39:1. In a particular embodiment of the invention, D₈:L₂ may be about 0.4:1.

FIGS. 10-13 show a catheter apparatus 300 in accordance with another embodiment of the present invention. The catheter apparatus 300 includes a shaft 302, and the shaft includes an intermediate portion 303 and an expanded distal tip 304. In this embodiment the distal tip 304 is substantially bulb-shaped. As used herein, the term “bulb-shaped” refers to an expanded distal tip having at least a portion of the inner and/or outer apertures 314 running radially outward from an inner wall 306 of the shaft to an outer wall 308 of the shaft, which may be used for diagnostic purposes such as aspirating or removing thromboembolic material or other particles from a blood vessel. The expanded distal tip 304 may optionally include a soft and substantially flexible atraumatic material 316 at the distal end 307 of the distal tip 304. This atraumatic material 316 may optionally be coated or constructed with a material of higher atomic density to aid in visualizing the distal tip 304, for instance, under fluoroscopy.

FIG. 10 shows that the catheter 300 may include a radially expandable segment, such as an inflatable balloon segment 318, disposed on the intermediate portion 303 of the shaft 302. FIG. 10 also shows that the intermediate portion 303 of the shaft 302 containing the inflatable balloon segment 318 may include one or more shaft apertures 320 to allow for the inflatable balloon segment 318 to be inflated and/or deflated. The intermediate portion 303 of the shaft 302 containing inflatable balloon segment 318 may also include one or more radiopaque markers 322 constructed with a material of higher atomic density to help show the location of the inflatable balloon segment 318 on the shaft 302.

The catheter shaft 302 also preferably includes an interior cavity defining a first lumen 324, of which a cross-sectional portion is shown in FIGS. 11-13, running inside the catheter 300 substantially from a proximal end (not shown) of the catheter 300 and extending substantially to the expanded distal tip 304 of the catheter 300. The first lumen 324 may be used to accommodate guide wires and/or other diagnostic devices or instruments. The catheter shaft 302 also may include an interior cavity defining a second lumen 328 running adjacent to the first lumen 324 substantially from the proximal end (not shown) of the catheter 300 and extending substantially to the intermediate portion 303 of the shaft 302 containing the inflatable balloon segment 318. This second lumen 328 may be used, for example, to provide gases, liquids, or other materials via the shaft apertures 320 to the inflatable balloon segment 318 for a purpose such as inflating and/or deflating the balloon segment.

FIG. 11 is a cross-sectional view of the intermediate portion 303 of the shaft 302 of the catheter 300 shown in FIG. 10 taken along the line 14-14. FIG. 11 shows that the shaft 302 includes inner wall 306 and outer wall 308. The cross section of the catheter 300 shown in FIG. 16 may have an inner diameter D₉ defined and measured with respect to the inner wall 306 of the shaft 302, an outer diameter D₁₀ defined and measured with respect to the outer wall 308 of the shaft 302, and a thickness T₅ defined as the distance between the inner wall 306 and the outer wall 308 of the shaft 302. The inner diameter D₉ may range from about 0.4 mm to about 0.6 mm, preferably from about 0.45 mm to about 0.55 mm. A particularly preferred diameter D₉ may be about 0.48 mm. The outer diameter D₁₀ may range from about 0.9 mm to about 3 mm, preferably from about 1.4 mm to about 3 mm. A particularly preferred diameter D₁₀ may be about 2.5 mm. The thickness T₅ may range from about 0.25 mm to about 1.2 mm, preferably from about 0.5 mm to about 1.2 mm. A particularly preferred thickness Ts may be about 1 mm. Although a particular cross-sectional piece of the intermediate portion 303 of the catheter shaft 302 is shown in FIG. 11, it is to be understood that the diameters D₉ and D₁₀, and the thickness T₅ may be measured at other locations along the intermediate portion of the catheter shaft, such as the intermediate portion 303 of the shaft 302 containing the inflatable balloon segment 318. The first lumen 324 is illustrated in the cross section of the shaft 302 shown in FIG. 11.

FIG. 12 illustrates a cross-sectional portion of the expanded distal tip 304 of the shaft 302 of the catheter apparatus 300 shown in FIG. 10 taken along the line 15-15, which is at the approximate midpoint 309 of the length of the expanded distal tip 304. In this embodiment, the cross-sectional diameter of the distal tip 304, measured with respect to the inner wall 306 and outer wall 308 of the shaft, gradually increases from a proximal end 305 of the expanded distal tip to approximately the midpoint 309 of the expanded distal tip, and then gradually decreases slightly from approximately the midpoint 309 of the expanded distal tip to the distal end 307 of the expanded distal tip 304. FIG. 12 shows that an inner diameter D₁₁ may be defined and measured with respect to the inner wall 306 of the shaft 302, an outer diameter D₁₂ may be defined and measured with respect to an outer wall 308 of the shaft 302, and a thickness T₆ may be defined as the distance between the inner wall 306 and the outer wall 308 of the shaft 302. The inner diameter D₁₁ may range from about 1 mm to about 1.5 mm, preferably from about 1.15 mm to about 1.3 mm. A particularly preferred diameter D₁₁ may be about 1.2 mm. The outer diameter D₁₂ may range from about 1.5 mm to about 3.8 mm, preferably from about 2.2 mm to about 3.5 mm. A particularly preferred diameter D₁₂ may be about 3.2 mm. The thickness T₆ may range from about 0.25 mm to about 1.2 mm, preferably from about 0.5 mm to about 1.1 mm. A particularly preferred thickness T₆ may be about 1.0 mm. The first lumen 324 is also illustrated in FIG. 12.

FIG. 13 shows a cross-sectional portion of the expanded distal tip 304 of the catheter 300 shown in FIG. 10 taken along the line 16-16, which is approximately at the distal end 307 of the expanded distal tip 304. As shown in FIG. 13, an inner diameter D₁₃ may be defined and measured with respect to the inner wall 306 of the shaft 302, an outer diameter D₁₄ may be defined and measured with respect to the outer wall 308 of the shaft 302, and a thickness T₇ may be defined as the distance between the inner wall 306 and the outer wall 308 of the shaft 302. The inner diameter D₁₃ may range from about 0.8 mm to about 1.2 mm, preferably from about 0.95 mm to about 1.1 mm. A particularly preferred diameter D₁₃ may be about 1 mm. The outer diameter D₁₄ may range from about 1.3 mm to about 3.6 mm, preferably from about 2 mm to about 3.3 mm. A particularly preferred diameter D₁₄ may be about 3 mm. The thickness T₇ may range from about 0.25 mm to about 1.2 mm, preferably from about 0.5 mm to about 1.1 mm. A particularly preferred thickness T₇ may be about 1.0 mm. The first lumen 324 is also shown in FIG. 13.

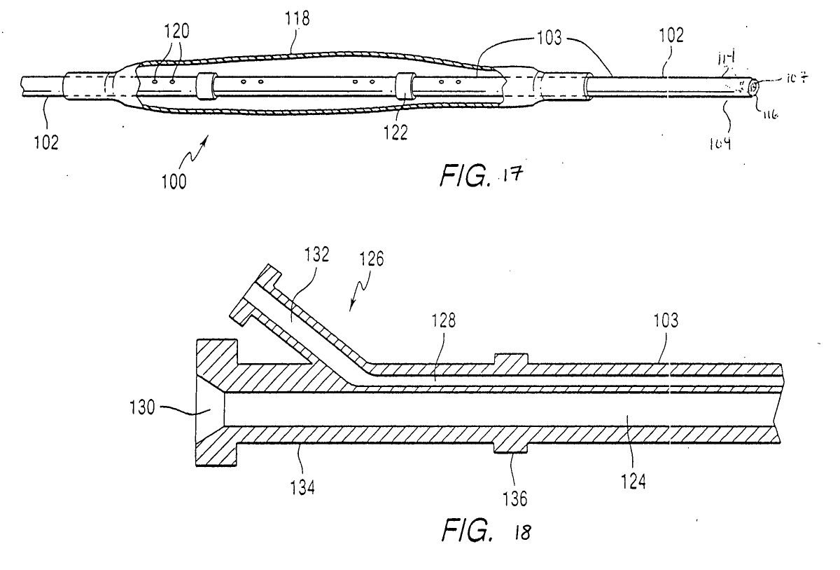

FIGS. 11 and 12 illustrate that the diameter D₁₁ of the expanded distal tip 304 is greater than the diameter D₉ of the intermediate portion 403 of the shaft 302, and that the diameter D₁₂ of the expanded distal tip 304 is greater than the diameter D₁₀ of the shaft 302. FIGS. 16 and 17 also illustrate that the thickness T₅ is substantially equal to the thickness T₆.

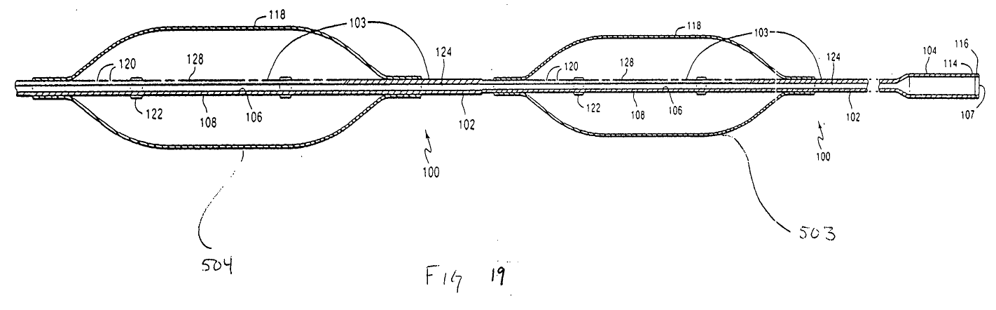

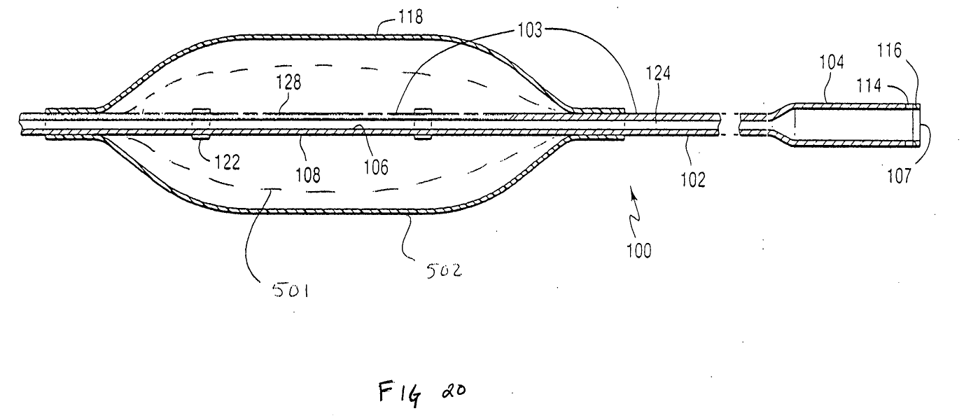

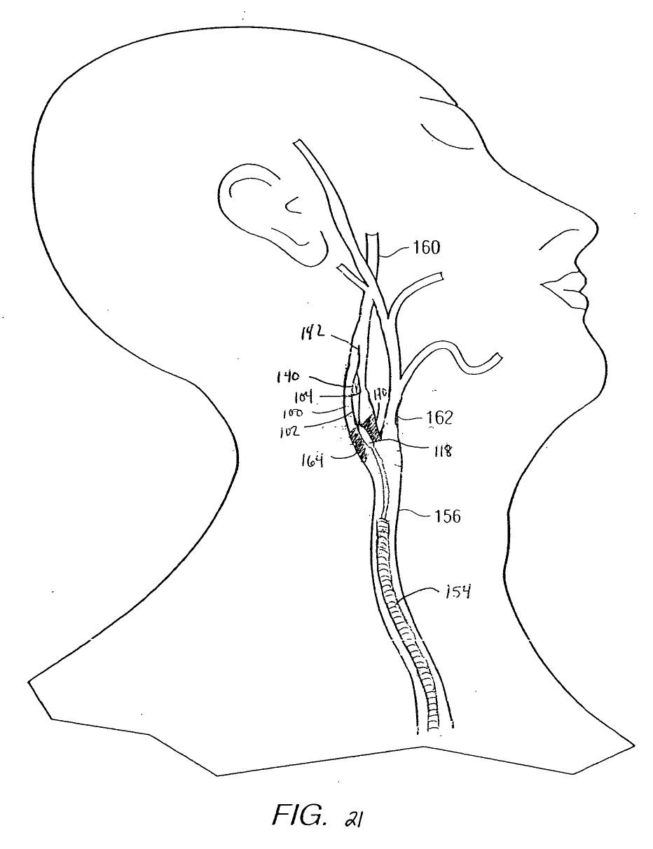

FIGS. 11 and 13 illustrate that the diameter D₁₃ of the expanded distal tip 304 is greater than the diameter D₉ of the shaft 302, and that the diameter D₁₄ of the expanded distal tip 304 is greater than the diameter D₁₀ of the shaft 302. FIGS. 16 and 18 also illustrate that the thickness T₅ is substantially equal to the thickness T₇.

In this embodiment, a ratio of the diameter D_{11 to D}₉ may be defined as D₁:D₉. D₁:D₉ may range from about 2.3:1 to about 2.5:1, preferably from about 2.4:1 to about 2.5:1. In a particularly preferred embodiment, D₁₁:D₉ may be about 2.5:1. In this embodiment, a ratio of the diameter D₁₂ to the diameter D₁₀ may also be defined as D₁₂:D₁₀. D₁₂:D₁₀ may range from about 1.3:1 to about 1.7:1, preferably from about 1.3:1 to about 1.6:1. In a particularly preferred embodiment, D₁₂:D₁₀ may be about 1.2:1.

FIG. 10 shows that the length of the expanded distal tip 304 may be defined as L₃. The length L₃ may range from about 0.3 cm to about 1 cm, preferably from about 0.5 cm to about 0.7 cm. A particularly preferred length L₃ may be about 0.7 cm. In this embodiment, a ratio of the diameter D₁ of the distal tip 304 to the length L₃ of the distal tip 304 may be defined as D₁:L₃. D₁:L₃ may range from about 0.38:1 to about 0.14:1, preferably from about 0.23:1 to about 0.19:1. In a particular embodiment, D₁:L₃ may be about 0.17:1. In this embodiment, a ratio of the diameter D₁₂ of the distal tip 304 to the length L₃ of the distal tip 304 may be defined as D₁₂:L₃. D₁₂:L₃ may range from about 0.5:1 to about 0.38:1, preferably from about 0.44:1 to about 0.5:1. In a particular embodiment of the invention, D₁₂:L₃ may be about 0.46:1.

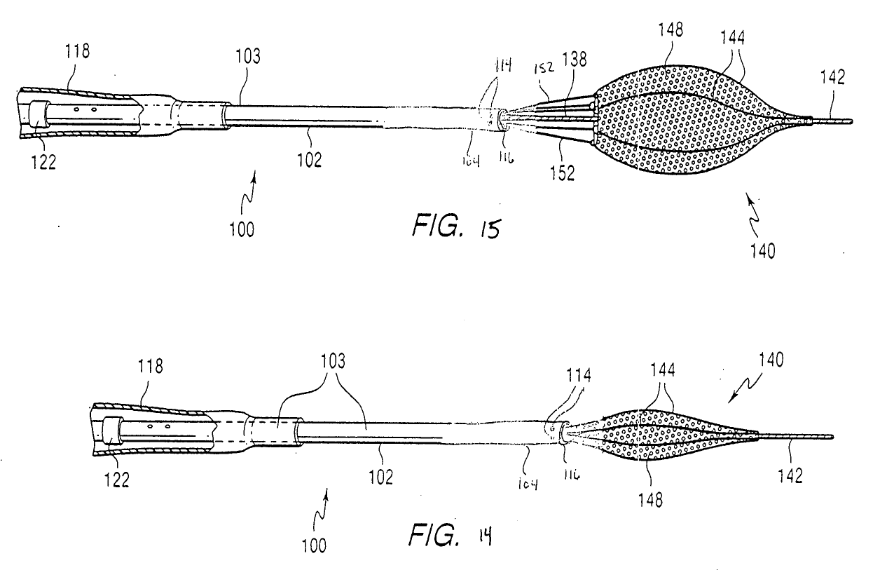

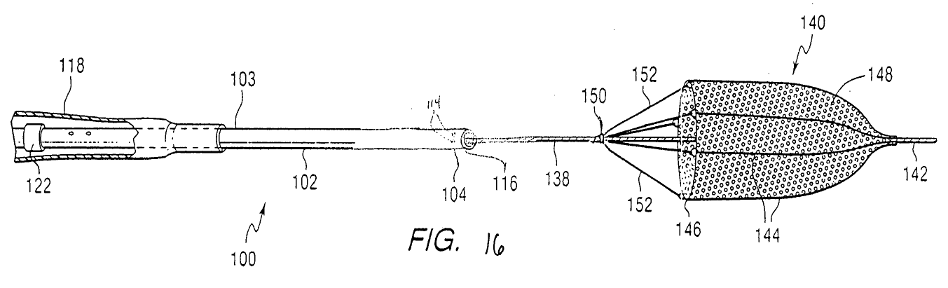

Once the delivery of the sheath to an area adjacent an occluded and/or stenotic area of a blood vessel is completed, the thromboembolic protection device can be released from the housing of the sheath. The thromboembolic protection device can be deployed from a collapsed position within the sheath towards an interior wall of a blood vessel to achieve an expanded position. In the expanded position, the thromboembolic protection device is capable of trapping embolic material typically broken loose by dilation or stenting of an occluded and/or stenotic portion of a blood vessel. As shown in FIGS. 14-16 to transition the thromboembolic protection device 140 from a collapsed position to an expanded position, the delivery sheath 104 may be removed from the embolic filter assembly, thereby allowing the resilient ribs 144 to naturally expand, in turn causing the embolic filter assembly 140 to open to a substantially expanded position. In one embodiment, the delivery sheath 104 may be “peeled” away from the embolic filter assembly 140 and removed from the patient using a string, cord, suture, or other appropriate peeling means. In another embodiment, the guide wire may be held in a substantially stationary position, and the introducer sheath may be slideably removed from the embolic filter assembly 140 and subsequently removed from the patient.

As shown in FIGS. 14-16, the guide wire 138 may be made of any suitable material, such as stainless steel, nickel titanium alloy (Nitinol), coiled spring stainless steel or other related alloys, and the first lumen 124 of the catheter apparatus 100 may be structured and arranged to receive the guide wire 138 within the first lumen 124. In one embodiment, the guide wire 138 may run substantially along the entire length of the first lumen 124, and a proximal end (not shown) of the guide wire 138 may protrude from the first port 130 of the first lumen 124. Although a guide wire is shown in this embodiment, other types of flexible tubing may also be used. The tubing or guide wire preferably has an outer diameter of greater than about 0.05 cm and less than 0.25 cm, however guide wires with other suitable diameters may be used. For example, the guide wire 138 may have an outer diameter of about 0.09 cm.

The embolic filter assembly 140 may be of any suitable construction for collecting and containing embolic material that is well known in the art. In one embodiment, as most clearly illustrated in FIG. 16, the embolic filter assembly 140 may include a plurality of ribs 144 spaced around the external circumference of the guide wire 138. More or less ribs may be used. For example, although four ribs are shown, a device with six ribs may be constructed, and the ribs may be spaced at various intervals around the circumference of the guide wire 138, for example, in an equiangular fashion. The ribs 144 are preferably formed of a resilient material, such as stainless steel, or Nitinol memory metal or plastic, which is pre-stressed or pre-formed resulting in an expandable or outward bias. The tips 146 of the ribs 144 may be preferably curved inward to minimize trauma to the blood vessel wall.

A filter material 148 spans the gaps between and is secured to the ribs 144. The filter material 148 is preferably a finely porous mesh capable of trapping embolic material broken loose from interventional procedures, but coarse enough to allow blood to pass through. Suitable filter materials include porous PTFE, fabrics and metals. When metal such as Nitinol memory metal is used as the filter material, it preferably has a low profile and facilitates trackability of the filter during use. The filter material 148 may be attached to the ribs 144 by any suitable means such as sutures, pockets, adhesives and the like. In one embodiment, the filter material 148 may be tied to the ribs 144 by sutures, which also may act as control strings of the embolic filter assembly 140.

In many medium sized blood vessels, the embolic filter assembly 140 may expand to a diameter against the wall of the vessel from about 4 mm to about 10 mm, often from about 6 mm to about 8 mm. In larger vessels such as the aorta, the embolic filter assembly 140 may expand to a diameter from about 10 mm to about 30 mm, often from about 12 mm to about 20 mm.

As most clearly illustrated in FIG. 16, the tips 146 of the ribs 144 may be attached to a collar 150 via a plurality of control strings 152. The control strings 152 may be made of any suitable material such as metal wires, sutures or suture-like materials. The diameter of each control string 152 is preferably 0.03 cm or less. The collar 150 is preferably in sliding engagement with the guide wire 138, so that the collar may move freely along the guide wire. In another embodiment, a collar is not used, and instead the control strings 152 may be attached directly to the guide wire 138 by any suitable means. In yet another embodiment, the embolic filter assembly 140 is rotatable about an axis, independent of the sheath 100 or guide wire 138.

In another embodiment of the invention, an embolic filter assembly and guide wire combination may be used with the present invention as disclosed in copending commonly owned U.S. patent application Ser. No. 09/476,829 filed Jan. 3, 2000, which is hereby incorporated by reference. In this embodiment, an embolic filter assembly may be substantially structured and arranged as described above, however, multiple control strings may be attached to an actuator located near a proximal end of a guide wire. The control strings may run inside the guide wire and may exit the guide wire through holes located in a collar, such as the collar 150 described above. The control strings may then be secured to the tips of a plurality of ribs of the embolic filter assembly. To open the embolic filter assembly, the actuator may be pushed forward, releasing tension upon the control strings and allowing the embolic filter assembly to self-expand. When the interventional procedure is complete, the actuator may be pulled, tensioning the control strings and causing the embolic filter assembly to retract, allowing the dislodged embolic material to be retained in a deep pocket formed by the filter material of the embolic filter assembly. The sheath apparatus 100 may then be advanced toward a distal end of the guide wire until the collapsed embolic filter assembly is safely stored within the distal tip 104 of the catheter apparatus 100, or the guide wire 138 may be pulled until the collapsed embolic filter assembly is safely stored within the distal tip 104 of the sheath apparatus 100.

In an embodiment of the present invention, the sheath can be circumferentially disposed about a guide wire. The guide wire can be inserted into a patient's blood vessel and the sheath and thromboembolic protection device releasably housed within the interior of the sheath can be subsequently advanced along the guide wire into the patient's blood vessel. In another embodiment, the guide wire, sheath and thromboembolic protection device can be introduced into the patient's blood vessel contemporaneously. In one embodiment of the present invention, the guide wire is capable of being steered or directed into and/or within the patient's blood vessel. The guide wire can also extend beyond the thromboembolic protection device and/or sheath in the direction of the patient's blood vessel to allow an operator to direct the thromboembolic protection device within the blood vessel.

As shown in FIGS. 1, 2 and 17, the sheath apparatus 100 includes a radially expandable initial dilation segment 118, such as an inflatable balloon segment, disposed on the intermediate portion 103 of the shaft 102. In one embodiment, the radially expandable initial dilation segment can comprise a plaque-smoothing balloon. The radially expandable initial dilation segment dilates the blood vessel by smoothing and/or flattening at least some of the jagged or rough edges of the deposits present on the interior wall of the blood vessel. The radially expandable initial dilation segment can be disposed on the delivery sheath in an area adjacent the section of the delivery sheath capable of housing the thromboembolic protection device, such as the distal tip. In another embodiment, the radially expandable initial dilation segment can be circumferentially disposed about the sheath.

The radially expandable initial dilation segment may have any desired deflated diameter and inflated diameter. For example, the radially expandable initial dilation segment can have a deflated diameter of about 1 mm and an inflated diameter of about 3 mm. FIGS. 1 and 2 show the initial dilation segment 118 in a substantially inflated position and FIG. 4 shows the initial dilation segment 118 in a substantially deflated position.

When the thromboembolic protection device is delivered to an area downstream of the occluded area, the radially expandable initial dilation segment can be inserted in its deflated state into the interior of the occluded area of the blood vessel. Once the expandable segment is inserted into the occluded area and the thromboembolic protection device is deployed, the radially expandable initial dilation segment can be inflated to an inflated state to dilate the blood vessel and smooth and/or flatten the jagged or rough edges of the deposits present on the interior walls of the blood vessel.

The initial dilation segment 118 may be made out of any suitable material capable of being expanded to a desired diameter within a blood vessel, such as but not limited to, PET, polyethylene, polyamide, PTFE, and/or other suitable materials that can exert a sufficient radial force to smooth and/or flatten the rough edges of debris and/or plaque present in an occluded and/or stenotic blood vessel. FIGS. 1, 2 and 17 also show that the intermediate portion 103 of the shaft 102 containing the initial dilation segment 118 may include one or more shaft apertures 120 to allow for the initial dilation segment 118 to be inflated and/or deflated. The intermediate portion 103 containing initial dilation segment 118 may also include one or more radiopaque markers 122 constructed with a material of higher atomic density to help show the location of the initial dilation segment 118 on the shaft 102.

As most clearly shown in FIGS. 1 and 18, the shaft 102 may include an interior cavity defining a first lumen 124 running inside the catheter 100 substantially from a proximal end 126 of the sheath 100, as shown in FIG. 18, and extending substantially to the distal tip 104 of the catheter 100 as shown in FIG. 1. The first lumen 124 may be used to accommodate guide wires and/or other diagnostic devices or instruments. As also shown in FIGS. 1 and 18, the shaft 102 also may include an interior cavity defining a second lumen 128 running adjacent to the first lumen 124 substantially from the proximal end 126 of the catheter 100, as shown in FIG. 18, and extending substantially to the intermediate portion 103 of the shaft 102 containing the initial dilation segment 118, as shown in FIG. 1. This second lumen 128 may be used, for example, to provide gases, liquids, and/or other materials via the shaft apertures 120 to the inflatable balloon segment 118 for the purposes of inflating or deflating the initial dilation segment.

FIG. 18 shows a proximal end 126 of the sheath or catheter apparatus 100. The proximal end 126 of the catheter 100 includes a first port 130 in flow communication with the first lumen 124, and a second port 132 in flow communication with the second lumen 128. The first port 130 and the second port 132 may both be substantially enclosed in a Y-shaped housing 134 as illustrated in FIG. 18. FIG. 18 shows that the Y-shaped housing 134 may also include a reinforcing lip or ridge 136 for providing the Y-shaped housing 134 with added structural support. As shown in FIG. 18, the Y-shaped housing 134 may be attached to the shaft 102 with any suitable fastening means, or optionally may be formed as an integral part of the catheter 100 during manufacture. The first port 130 may be used to supply the first lumen 124 with guide wires, suction for aspirating embolic material, and/or other diagnostic instruments, and the second port 132 may be used to supply the second lumen 128 with materials for inflating and deflating the inflatable balloon segment 118, such as, but not limited to, various gases and liquids.

In one embodiment, after inflation, the radially expandable initial dilation segment may be deflated and the combined delivery sheath and radially expandable initial dilation segment may be removed from the occluded or stenotic area along the guide wire leaving the thromboembolic protection device deployed.

In another embodiment, prior to removal of the delivery sheath, a stent may be advanced along the guide wire to the occluded area using any conventional stent delivery means. In another embodiment, the stent may be delivered on the delivery sheath to the occluded and/or stenotic area along with the radially expandable initial dilation segment. In yet another embodiment, the stent can be circumferentially disposed about the radially expandable initial dilation segment and positioned within the interior of the occluded area of the blood vessel by the delivery sheath.

In certain embodiments, a radially expandable stent expanding segment, such as an inflatable balloon, can be disposed on the delivery sheath in an area adjacent the radially expandable initial dilation segment. The radially expandable stent expanding segment may have any desired deflated diameter and inflated diameter. For example, the radially expandable stent expanding segment may have a deflated diameter of about 1 mm and an inflated diameter of from about 5 mm to about 6 mm. Once the radially expandable stent expanding segment is positioned within the stent, the segment may be inflated to an inflated position, thereby expanding the walls of the stent substantially flush with the walls of the blood vessel. Once the walls of the stent are expanded to their expanded position, the occluded area of the blood vessel becomes dilated. The radially expandable stent expanding segment may subsequently be deflated.

In certain embodiments, once the radially expandable stent expanding segment has been deflated and the walls of the stent have been expanded, the delivery sheath of the present invention may be removed from the patient's blood vessel. In another embodiment, the delivery sheath may optionally be advanced further along the guide wire until the expanded thromboembolic protection device substantially meets the distal tip of the catheter. The thromboembolic protection device may then be collapsed and pulled into the distal tip of the delivery sheath via the guide wire, or the delivery sheath may be advanced further along the guide wire until the collapsed protection device is sufficiently stored within the distal tip. In this embodiment, the distal tip of the sheath has a volume that is capable of safely and effectively storing the thromboembolic protection device filled with embolic material. The sheath and the collapsed thromboembolic protection device may then safely be removed from the blood vessel of the patient together, as a unit. Accordingly, in one embodiment of the present invention, the process for opening occluded or stenotic blood vessels requires fewer steps than traditional processes.

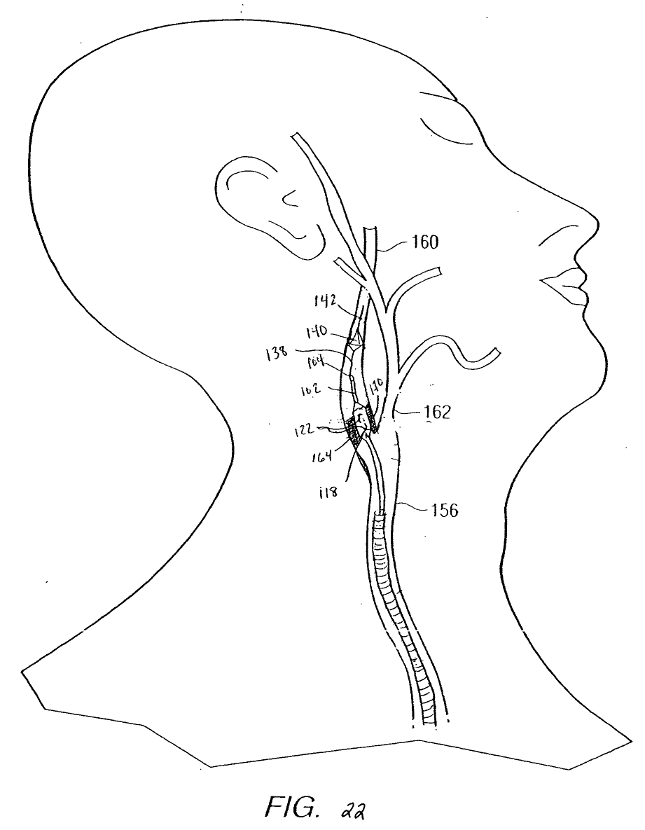

In another embodiment, as shown in FIG. 19, a single sheath comprises two radially expandable segments in tandem. In one embodiment a radially expandable initial dilation segment 503 and a radially expandable stent expanding segment 504 are each disposed on the shaft of the delivery sheath. In this embodiment, the initial dilation segment 503 may be located adjacent the distal tip, or at a distal location of the shaft. Once the initial dilation segment 503 has been inflated, dilated at least a portion of the stenoted and/or occluded area of the blood vessel, and subsequently deflated, the initial dilation segment 503 can be advanced through the stenoted and/or occluded area and the stent expanding segment 504 can be positioned within the stenoted and/or occluded area. The stent may be introduced into the stenoted and/or occluded area of the blood vessel before or after the stent-expanding segment is positioned within the occluded area. The stent may also be positioned within the occluded area simultaneously with the stent-expanding segment.

In another embodiment, as shown in FIG. 20, the delivery sheath comprises a single dual-stage radially expandable segment, such as an inflatable balloon, disposed on the shaft that may perform the functions of both the radially expandable initial dilation segment and the radially expandable stent expanding segment. In this embodiment, the dual-stage radially expandable segment is first inserted into the occluded and/or stenoted area and expanded to a first inflation level 501 to dilate and/or smooth at least a portion of the stenoted and/or occluded area. In one embodiment, the first inflation level 501 has an expanded diameter sufficient to perform the function of the initial dilation segment, such as about 3 mm. After the dual-stage segment has been inflated to the first inflation level 501, the expandable segment may be deflated and a stent may be introduced into the stenoted and/or occluded area on the exterior of the dual-stage segment. In another embodiment, the dual-stage segment can remain substantially inflated when the stent is introduced to the occluded and/or stenotic area. In yet another embodiment, the dual-stage segment is removed from the occluded area, the stent is introduced into the occluded area, and the dual-stage segment is inserted into the interior of the stent. Once the stent and dual-stage segment are both located within the interior of the occluded area, the dual-stage segment is subsequently inflated to a second inflation level 502 sufficient to expand the walls of the stent flush against the interior walls of the blood vessel, such as about 5 to 6 mm. After the stent has achieved its expanded position, the dual-stage segment can be deflated. As shown in FIG. 20, in another embodiment, the initial inflation level 501 can also be achieved by disposing a second radially expandable segment within the radially expandable segment 118.

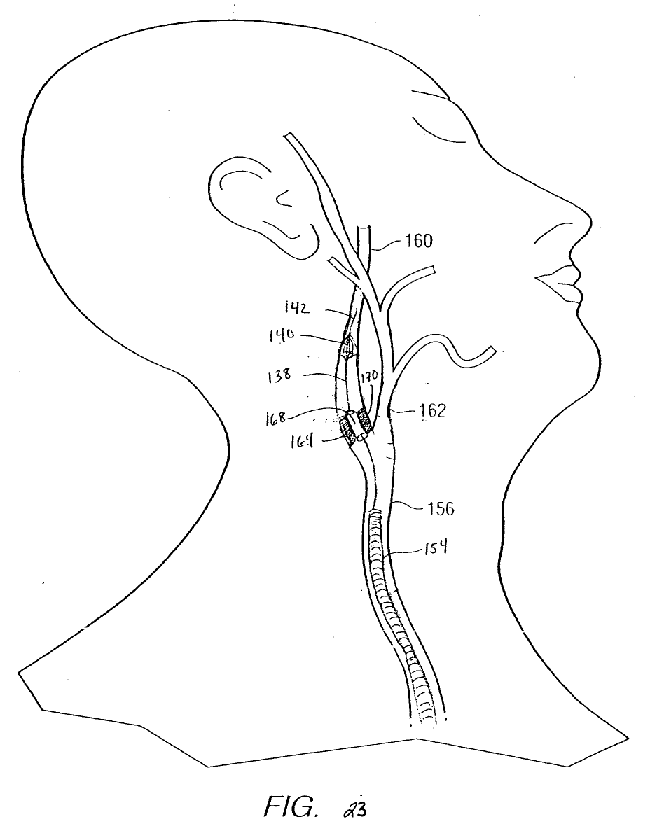

Methods of inserting and deploying the delivery sheath of the present invention in accordance with embodiments of the present invention are shown in FIGS. 21-24.

As illustrated in FIG. 21, a guiding sheath 154 may be inserted into a blood vessel such as a common carotid artery 156 located proximal to a bifurcation 158 between an internal carotid artery 160 and an external carotid artery 162. A guide wire 138 containing an embolic filter assembly 140 housed in the distal tip 104 of a delivery sheath 100 and having an initial dilation segment 118, such as a plaque-smoothing balloon, disposed on the shaft 102 may be advanced through the guiding sheath 154 and past a stenotic section 164 of the internal carotid artery 160 that is affected by stenosis, with the embolic filter assembly 140 in a substantially collapsed position.

As shown in FIG. 22, the embolic filter assembly 140 may then be opened or expanded against the interior walls of the blood vessel. Initial dilation segment 118 may then be inflated by supplying any suitable gas or liquid to the initial dilation segment 118 via the second port 132, second lumen 128, and shaft apertures 120. As the initial dilation segment 118 is substantially inflated, the stenotic section 164 of the internal carotid artery 160 preferably will become smoothed so that delivery of a stent 168 to the stenotic area is possible without concern that the stent 168 will become caught on a jagged edge of plaque or other material deposited on the interior of the blood vessel wall. As the stenotic section 164 is smoothed, pieces of stenotic material and other embolic material may become dislodged and may flow through the internal carotid artery 160 and be captured by the expanded embolic filter assembly 140. Once the initial dilation segment 118 has smoothed the surface of the stenotic area, the initial dilation segment 118 is deflated by removing the gas or liquid used to inflate the initial dilation segment 118.

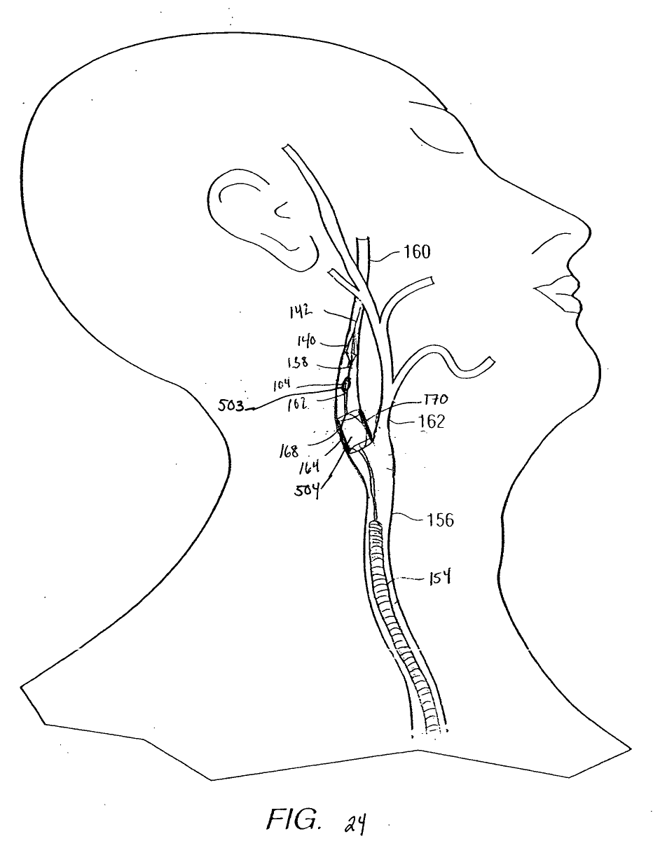

As shown in FIG. 23, a vascular stent 168 may then be deployed via the guide wire 138 to the location of the stenosis 166. Any suitable type of stent may be used, typically a self-expanding stent that presses against the stenosis once it is deployed is used in conjunction with the present invention.

In another embodiment, as shown in FIG. 24, the stent expanding segment 504 of the present invention may be positioned along the guide wire 138 so that the stent-expanding segment 504 is substantially lined up with the stent 168 and the stenotic section 164 of the internal carotid artery 160. Radiopaque markers 122 located at a portion of the shaft 102 containing the stent-expanding balloon segment 504 may aid in positioning the stent-expanding segment 504 within the interior of the stent 168 and the stenotic section 164 of the internal carotid artery 160. The stent-expanding segment 504 may then be substantially inflated by supplying any suitable gas or liquid to the stent-expanding segment 504 via the second port 132, second lumen 128, and shaft apertures 120. As the stent-expanding segment 504 is substantially inflated, the stenotic section 164 of the internal carotid artery 160 preferably will become dilated and the stent will preferably become effectively embedded into the wall 170 of the internal carotid artery 160. As the stenotic section 164 of the internal carotid artery 160 is dilated with the inflatable stent expanding segment 504, pieces of stenotic material and other embolic material may become dislodged and may flow through the internal carotid artery 160 and be captured by the expanded embolic filter assembly 140.

Once the vascular stent 168 is in place, the inflatable stent expanding segment 504 may be substantially deflated via the second port 132, second lumen 128, and shaft apertures 120, and the recovery sheath 100 may be further advanced coaxially along the guide wire 138 towards the distal end 142 of the guide wire 138. Alternatively, the guide wire 138 may be retracted towards the distal tip 104 of the recovery sheath 100.

It will be appreciated that the catheter apparatus 200 shown in FIGS. 7-9 and the catheter apparatus 300 shown in FIGS. 10-13 operate in substantially the same manner as described above.

Whereas particular embodiments of this invention have been described above for purposes of illustration, it will be evident to those skilled in the art that numerous variations of the details of the present invention may be made without departing from the invention.

1. A vascular catheter for delivery of a thromboembolic protection device within a blood vessel of a patient comprising:
a shaft including an expanded distal tip having an interior structured and arranged to releasably house at least a portion of the thromboembolic protection device within the interior of the distal tip during delivery of the thromboembolic protection device; and
a radially expandable initial dilation segment disposed on the shaft.

2. The vascular catheter of claim 1, wherein the shaft comprises a lumen running longitudinally inside the shaft extending substantially to the distal tip, and further comprising a guide wire in the lumen.

3. The vascular catheter of claim 2, wherein the thromboembolic protection device is attached to the guide wire.

4. The vascular catheter of claim 1, wherein the radially expandable initial dilation segment is expandable to a diameter of from 1 mm to 3 mm.

5. A vascular catheter for delivery of a thromboembolic protection device in a blood vessel of a patient comprising:
means for releasably housing at least a portion of the thromboembolic protection device during delivery of the thromboembolic protection device; and
means for initially dilating at least a portion of a stenoted and/or occluded area of the blood vessel.

6. The vascular catheter of claim 5, wherein the means of releasably housing includes an expanded distal tip.

7. The vascular catheter of claim 5, wherein the means for initially dilating is radially expandable to a diameter of from 1 mm to 3 mm.

8. A method of dilating a blood vessel, comprising:
inserting into the blood vessel a vascular catheter comprising a shaft, a thromboembolic protection device housed at least partially within a distal tip of the shaft, and a radially expandable initial dilation segment;
expanding the thromboembolic protection device from the shaft from a collapsed position to an expanded position; and
expanding the radially expandable initial dilation segment to dilate at least a portion of a stenoted and/or occluded area of the blood vessel.

9. The method of claim 8, further comprising inserting a guide wire disposed within the shaft and attached to the thromboembolic protection device into the blood vessel.

10. A vascular catheter comprising:
a shaft structured and arranged to at least partially house a thromboembolic protection device;
a radially expandable initial dilation segment disposed on the shaft; and
a radially expandable stent expanding segment disposed on the shaft.

11. The vascular catheter of claim 10, wherein the shaft further comprises an expanded distal tip having an interior structured and arranged to releasably house at least a portion of the thromboembolic protection device within the distal tip.

12. The vascular catheter of claim 10, wherein the shaft comprises a lumen running longitudinally inside the shaft, and further comprising a guide wire in the lumen.

13. The vascular catheter of claim 10, wherein the radially expandable initial dilation segment and the radially expandable stent expanding segment are disposed at different longitudinal positions along a length of the shaft.

14. The vascular catheter of claim 13, wherein the radially expandable initial dilation segment is disposed at a distal location along a length of the shaft with respect to the radially expandable stent expanding segment.

15. The vascular catheter of claim 10, wherein at least one of the radially expandable initial dilation segment and the radially expandable stent expanding segment is radially disposed within the other of the radially expandable initial dilation segment and the radially expandable stent expanding segment on the shaft.

16. The vascular catheter of claim 10, wherein the radially expandable initial dilation segment and the radially expandable stent expanding segment is a dual-stage radially expandable segment.

17. A vascular catheter comprising a shaft including:
means for initially dilating a portion of a stenoted and/or occluded area of the blood vessel; and
means for radially expanding a stent disposed about the shaft.

18. The vascular catheter of claim 17, wherein the means for initially dilating and the means for radially expanding a stent are disposed at different longitudinal positions along a length of the shaft.

19. The vascular catheter of claim 17, wherein one of the means for initially dilating and the means for radially expanding a stent is radially disposed within the other of the means for initially dilating and the means for radially expanding about the shaft.

20. The vascular catheter of claim 17, wherein the means for initially dilating and the means for radially expanding comprise a single dual-stage radially expandable segment.

21. A method of dilating a blood vessel comprising:
inserting into the blood vessel a vascular catheter comprising a shaft housing a thromboembolic protection device, a radially expandable initial dilation segment disposed on the shaft, and a radially expandable stent expanding segment disposed on the shaft;
expanding the thromboembolic protection device from the shaft;
expanding the radially expandable initial dilation segment to dilate at least a portion of a stenoted and/or occluded area of the blood vessel; and
expanding the radially expandable stent expanding segment to expand a stent against the stenoted and/or occluded area of the blood vessel.

22. The method of claim 21 wherein the thromboembolic protection device is expanded from the shaft before the initial dilation segment is expanded.

23. The method of claim 21, wherein the stent is positioned at the stenoted and/or occluded area of the blood vessel after the initial dilation segment is expanded.
